(19) United States
(12) Patent Application Publication
(10) Pub. No.: US 2024/0024909 A1
(43) Pub. Date: Jan. 25, 2024

(54) DIRECTABLE MIST-DELIVERY DEVICE AND REPLACEABLE BOTTLE THEREFOR

(71) Applicant: Oron ZACHAR, Tel Aviv (IL)

(72) Inventor: Oron ZACHAR, Tel Aviv (IL)

(21) Appl. No.: 18/037,060

(22) PCT Filed: Sep. 8, 2020
(86) PCT No.: PCT/IB2020/058342

§ 371 Date: May 15, 2023

(51) Int. Cl. B05B 17/06; B05B 17/00; B05B 15/68; B05B 7/08

(52) U.S. Cl. CPC B05B 17/0676; B05B 17/0646; B05B 15/68; B05B 7/0815

(57) ABSTRACT

A non-thermal mist-delivery device comprises a replaceable bottle system comprising (i) a bottle comprising an internal liquid-storage volume for holding a liquid, and (ii) a cap comprising a piezo assembly including an ultrasonically vibrable mesh membrane, a housing shaped to hold the replaceable bottle system therewithin, the housing comprising a fan, an air inlet and an annular air outlet, the inlet and the outlet defining an airflow path circumventing the replaceable bottle; control circuitry operative to electrically activate the fan and the piezo assembly in response to a user input, respectively to generate an airflow and to non-thermally deliver, via the aerosol outlet, a mist comprising droplets of the liquid.

### CROSS-REFERENCE TO RELATED APPLICATIONS

This patent application claims the benefit of U.S. Provisional Patent Application No. 62/897,340 filed on Sep. 8, 2019, and of U.S. Provisional Patent Application No. 62/993,884 filed on Mar. 24, 2020, both of which are incorporated herein by reference in their entirety.

### FIELD OF THE INVENTION

The present invention relates to mist-delivery devices and refillable and/or replaceable bottle systems for use therein, and to methods for using such devices. In particular the present invention relates to devices that use a fan-generated airflow to enable directable mist delivery.

### SUMMARY

According to embodiments disclosed herein, an externally-powered bottle system for use in a non-thermal mist delivery device comprises: (a) a bottle comprising an internal liquid-storage volume and a neck-aperture; (b) a cap configured for reversible engagement with the bottle, the cap having a smaller volume than the bottle and comprising (i) a piezo assembly including a sub-50-micron-mesh ultrasonically-vibrable mesh membrane, and (ii) an exposed electrical contact connected to the piezo assembly for receiving electrical power from an external source to activate the piezo assembly; and (c) a capillary pathway for conveying a liquid by capillary action, wherein when the bottle system is in an assembled state: (i) the cap is secured to the bottle so as to position the electrical contact on an externally accessible surface of the assembled bottle system and to create a water-tight seal around the perimeter of the neck-aperture such that the bottle system is water-tight when held in any orientation, and not water-tight when the bottle system is shaken, (ii) a proximal portion of the capillary pathway is restrained so as to be held in contact with an inwardly-facing surface of the mesh membrane or displaced therefrom by no more than 1 mm, and (iii) a distal portion of the capillary pathway is disposed within the liquid-storage volume so as to be in contact with a liquid disposed in the liquid-storage volume, such that when the liquid-storage volume is at least 30% full and the bottle system is in a vertical position or rotated from a vertical position by up to 60°, the capillary pathway is effective to convey a portion of the liquid to the mesh membrane for non-thermal production thereby of a mist comprising droplets of the liquid when the piezo assembly is electrically activated by delivery of electricity to the exposed electrical contact.

In some embodiments, the mesh membrane can comprise a sub-30-micron mesh. In some embodiments, the mesh membrane can comprise a sub-10-micron mesh.

In some embodiments, the capillary pathway can be disposed within the bottle such that a center of a proximal-most 10% portion of the capillary pathway is closer to a central axis of the bottle than a center of a distal-most 10% portion of the capillary pathway.

In some embodiments, the cap can comprise a fluid conveyance having a one-way valve, provided such that when the cap is secured to the bottle, the conveyance is effective to allow ingress of a liquid into the bottle and to preclude egress of the liquid from the bottle.

In some embodiments, when the bottle system is in an assembled state and the cap is secured to the bottle, a central axis of the bottle can pass through the mesh membrane.

In some embodiments, the bottle can include a solid-phase biologically-active material for being dissolved or suspended in droplets of an aqueous liquid misted by the piezo assembly. In some embodiments, the bottle can include a compartment for storing a solid-phase material, the compartment being in fluid communication with the liquid-storage volume.

In some embodiments, the securing of the cap to the bottle can be reversible.

In some embodiments, the neck-aperture of the bottle and an inlet portion of the cap can be correspondingly threaded such that securing the cap to the bottle can be accomplished by screwing one into the other. In some embodiments, removing the secured cap from the bottle can be accomplished without tools by applying a maximum torque of no more than 2.5 N·m.

In some embodiments, the neck-aperture of the bottle and an inlet portion of the cap can be configured to snap together so as to secure the cap to the bottle, at least one of the neck-aperture and the inlet portion including a snap-connector feature. In some embodiments, the neck-aperture of the bottle and an inlet portion of the cap, when in the assembled state, can be reversibly held together by static friction. In some embodiments, removing the secured cap from the bottle can be accomplished without tools by applying a maximum separating force of no more than 25 N.

According to embodiments disclosed herein, a non-thermal mist-delivery device, comprises: (a) a replaceable bottle system comprising (i) a bottle comprising an internal liquid-storage volume for holding a liquid and a neck aperture for introducing a liquid therethrough into the liquid-storage volume at ambient pressure, (ii) a cap comprising a piezo assembly including an ultrasonically vibrable mesh membrane, the cap configured to be reversibly secured to the bottle to create a waterproof seal between the cap and the neck aperture of the bottle, (iii) a capillary pathway for conveying a portion of the liquid by capillary action from the liquid-storage volume to the mesh membrane, and (iv) a bottle-system-electrical-contact connected to the piezo assembly and disposed on an exposed surface of the bottle system; (b) a housing comprising a powered fan, an air inlet and an annular air outlet, an aerosol outlet, and a housing-electrical-contact, the housing shaped to stably hold the replaceable bottle system oriented therewithin such that the bottle-electric-contact is in contact with the housing-electric-contact and the mesh membrane faces the aerosol outlet; (c) a base for supporting the housing, the base comprising a pivot about which the housing can be caused to pivot through a pivot-range of at least 60°; (d) control circuitry operative to electrically activate the fan and the piezo assembly in response to a user input, respectively to generate an airflow and to non-thermally deliver, via the aerosol outlet, a mist comprising droplets of the liquid held in the liquid-storage volume; and (e) a power supply for powering the fan and the piezo assembly, wherein when the bottle system is in an assembled state: (i) the cap is secured to the bottle so as to create a water-tight seal around the perimeter of the neck-aperture such that the bottle system is water-tight when held in any orientation, and not water-tight when the bottle system is shaken, (ii) a proximal portion of the capillary pathway is restrained so as to be held in contact with an inwardly-facing surface of the mesh membrane or displaced therefrom by no more than 1 mm, and (iii) a distal portion of the capillary pathway is disposed within the liquid-storage volume so as to be in contact with a liquid disposed in the liquid-storage volume, such that when the liquid-storage volume is at least 30% full and the bottle system is in a vertical position or rotated from a vertical position by up to 60°, the capillary pathway is effective to convey a portion of the liquid to the mesh membrane for non-thermal production thereby of a mist comprising droplets of the liquid when the piezo assembly is electrically activated by delivery of electricity to the exposed electrical contact.

In some embodiments, the neck-aperture of the bottle and an inlet portion of the cap can be correspondingly threaded such that securing the cap to the bottle can be accomplished by screwing one into the other. In some embodiments, removing the secured cap from the bottle can be accomplished without tools by applying a maximum torque of no more than 2.5 N·m.

In some embodiments, the neck-aperture of the bottle and an inlet portion of the cap can be configured to snap together so as to secure the cap to the bottle, at least one of the neck-aperture and the inlet portion including a snap-connector feature. In some embodiments, the neck-aperture of the bottle and an inlet portion of the cap, when in the assembled state, can be reversibly held together by static friction. In some embodiments, removing the secured cap from the bottle can be accomplished without tools by applying a maximum separating force of no more than 25 N.

In some embodiments, the cap can comprise a fluid conveyance for introducing a liquid into the liquid-storage volume, the conveyance being configured to preclude egress of the liquid from the bottle.

In some embodiments, the capillary pathway can be attached to the cap such that its assembly in and/or disassembly from the bottle system is together with the cap.

In some embodiments, when the bottle system is in an assembled state and the cap is secured to the bottle, a central axis of the bottle can pass through the mesh membrane.

In some embodiments, the air inlet and annular air outlet can collectively define an airflow path passing through the fan and circumventing the replaceable bottle.

In some embodiments, the fan-generated airflow exiting the annular air outlet can be effective to entrain a portion of the mist and thereby constrain lateral dispersion of the mist.

In some embodiments, the portion of the mist entrained by the generated airflow can be directable by pivoting the mist-delivery device.

In some embodiments, the fan-generated airflow exiting the annular air outlet can surround the mist.

According to embodiments disclosed herein, a bottle system can have any or all of the features disclosed hereinabove in any combination.

A method is disclosed, according to embodiments, for non-thermal delivery of a mist. The method comprises: (a) providing a bottle system comprising (i) a bottle having an internal liquid-storage volume for holding a liquid, (ii) a cap comprising a piezo assembly including a sub-50-micron ultrasonically-vibrable mesh membrane, and (iii) a capillary pathway for conveying a liquid by capillary action from the liquid-storage volume to the mesh membrane; (b) introducing an aqueous liquid to the liquid-storage volume, at ambient pressure, through a neck-aperture of the bottle; (c) securing the cap to the bottle to create a water-tight seal between the cap and the neck aperture of the bottle such that the bottle system is water-tight when held in any orientation, and not water-tight when the bottle system is shaken; (d) inserting the bottle system into a plenum of a housing of a mist-delivery device, the mist-delivery device comprising (i) a powered fan, (ii) a power supply for powering the fan and the piezo assembly, (iii) a base for supporting the housing, the base comprising a pivot about which the housing can be caused to pivot through a pivot-range of at least 60°, (iv) control circuitry operative to electrically activate the fan and the piezo assembly in response to a user input; (e) activating the device to deliver electricity from a power supply to the fan and to the piezo assembly, thereby causing the mesh membrane to non-thermally deliver a mist and causing the fan to generate an airflow, and (f) directing the fan-generated airflow by pivoting the mist-delivery device on a support comprising a pivot, wherein the bottle system is inserted in the housing in an assembled state such that: (i) a proximal portion of the capillary pathway is restrained so as to be held in contact with an inwardly-facing surface of the mesh membrane or displaced therefrom by no more than 1 mm, and (ii) a distal portion of the capillary pathway is disposed within the liquid-storage volume so as to be in contact with a liquid disposed in the liquid-storage volume, such that when the liquid-storage volume is at least 30% full and the bottle system is in a vertical position or rotated from a vertical position by up to 60°, the capillary pathway is effective to convey a portion of the liquid to the mesh membrane for non-thermal production thereby of a mist comprising droplets of the liquid when the piezo assembly is electrically activated.

In some embodiments, the neck-aperture of the bottle and an inlet portion of the cap can be correspondingly threaded such that securing the cap to the bottle can be accomplished by screwing one into the other.

In some embodiments, the neck-aperture of the bottle and an inlet portion of the cap can be configured to snap together so as to secure the cap to the bottle, at least one of the neck-aperture and the inlet portion including a snap-connector feature.

In some embodiments, the neck-aperture of the bottle and an inlet portion of the cap, when in the assembled state, can be reversibly held together by static friction.

In some embodiments, the electricity delivered from the power supply to the piezo assembly can flow through an electrical contact disposed on an external surface of the cap of the bottle system.

In some embodiments, the housing can comprise an air inlet at a first end of the plenum, and an annular air outlet at a second end of the plenum, the inlet and outlet defining an airflow path circumventing the inserted bottle system.

In some embodiments, the fan-generated airflow exiting the annular air outlet surrounds the mist can entrain a portion of the delivered mist and thereby constrains lateral dispersion of the mist.

In some embodiments, the method can additionally comprise: pivoting the mist-delivery device to direct the portion of the mist entrained by the generated airflow.

According to embodiments disclosed herein, a non-thermal mist-delivery device comprises: (a) a replaceable bottle system comprising (i) a bottle comprising an internal liquid-storage volume for holding a liquid, and (ii) a cap comprising a piezo assembly including an ultrasonically vibrable mesh membrane, (b) a housing shaped to hold the replaceable bottle system therewithin, the housing comprising a fan, an air inlet and an annular air outlet, the inlet and the outlet defining an airflow path circumventing the replaceable bottle; and (c) control circuitry operative to electrically activate the fan and the piezo assembly in response to a user input, respectively to generate an airflow and to non-thermally deliver, via the aerosol outlet, a mist comprising droplets of the liquid.

In some embodiments, the cap can be configured to be secured to the bottle.

In some embodiments, the mist-delivery device can additionally comprise a power supply for powering the fan and the piezo assembly.

In some embodiments, the replaceable bottle system can additionally comprise a capillary pathway for conveying a portion of the liquid by capillary action from the liquid-storage volume to the mesh membrane.

In some embodiments, the fan-generated airflow exiting the annular air outlet can be effective to entrain a portion of the mist and thereby constrain lateral dispersion of the mist.

In some embodiments, the replaceable bottle system can additionally comprise a bottle-system-electrical-contact connected to the piezo assembly and disposed on an exposed surface of the bottle system.

In some embodiments, the housing can additionally comprise a housing-electrical-contact, and the housing is shaped to stably hold the replaceable bottle system oriented therewithin such that the bottle-electric-contact is in contact with the housing-electric-contact and the mesh membrane faces the aerosol outlet.

In some embodiments, the mist-delivery device can additionally comprise a base for supporting the housing, the base comprising a pivot about which the housing can be caused to pivot through a pivot-range of at least 60°.

In some embodiments, the mist-delivery device can additionally comprise control circuitry operative to electrically activate the fan and the piezo assembly in response to a user input, respectively to generate an airflow and to non-thermally deliver, via the aerosol outlet, a mist comprising droplets of the liquid held in the liquid-storage volume.

In some embodiments, when the bottle system is in an assembled state and the cap is secured to the bottle, a central axis of the bottle can pass through the mesh membrane.

### BRIEF DESCRIPTION OF THE DRAWINGS

FIGS. 1A, 1B and 1C are schematic views of a bottle for use in a bottle system of a mist-delivery device, having an interior liquid-storage compartment and an aperture, according to embodiments of the present invention.

FIGS. 2A, 2B, 2C and 2D are schematic views of a cap for use in a bottle system of a mist-delivery device having a piezo assembly including an ultrasonic mesh membrane, according to embodiments of the present invention.

FIGS. 3, 4 and 5 are perspective views of bottle systems according to various embodiments of the present invention.

FIG. 6 is a schematic perspective view of a bottle and a piezo assembly engaged with a capillary pathway that is biased toward the mesh membrane of the piezo assembly, according to embodiments of the present invention.

FIG. 7 is a schematic perspective view of a bottle and a piezo assembly engaged with a capillary pathway partially enclosed in a holder, a solid-phase biologically-active material disposed within the bottle, according to embodiments of the present invention.

FIGS. 8A, 8B and 8C are schematic perspective views of bottles and piezo assemblies engaged with respective capillary pathways, according to various embodiments of the present invention.

FIG. 9A shows a bottle-system housing for a mist-delivery device according to embodiments of the present invention.

FIG. 9B is a schematic cutaway view of the bottle-system housing of FIG. 9A.

FIG. 10 is a schematic perspective cutaway view of the bottle-system housing of FIG. 9A, showing the placement therein of a bottle system and a fan.

FIG. 11 is a schematic cutaway view of the bottle-system housing of FIG. 9, showing a path of a fan-generated airflow, according to embodiments of the present invention.

FIG. 12 is a schematic perspective view of the bottle-system housing of FIG. 9, showing an annular airflow surrounding a mist generated by a piezo assembly, according to embodiments of the present invention.

FIG. 13 is an elevation view of a mist-delivery device according to embodiments of the present invention.

FIG. 14 is a schematic view of the mist-delivery device of FIG. 14, showing the egress of a mist and an airflow according to embodiments of the present invention.

FIGS. 15A and 15B are schematic views of a bottle for use in a bottle system of a mist-delivery device, according to embodiments of the present invention.

FIG. 16 is a schematic view of a cap for use in a bottle system of a mist-delivery device having a piezo assembly including an ultrasonic mesh membrane, according to embodiments of the present invention.

FIG. 17 is a schematic elevation view of a bottle system according to embodiments of the present invention.

FIG. 18 shows a bottle-system housing for a mist-delivery device according to embodiments of the present invention.

FIG. 19 is a schematic view of the bottle-system housing of FIG. 9A with a bottle system therein, according to embodiments of the present invention.

FIG. 20 is a schematic perspective view of the bottle-system housing of FIG. 19, showing an annular airflow surrounding a mist generated by a piezo assembly, according to embodiments of the present invention.

FIG. 21A is a schematic elevation view of a mist-delivery device according to embodiments of the present invention.

FIG. 21B is a schematic cutaway view of a bottom compartment of the base of the mist-delivery device of FIG. 21A, according to embodiments of the present invention.

FIGS. 22A and 22B show flowcharts of methods for non-thermal delivery of a mist, according to embodiments of the present invention.

### DETAILED DESCRIPTION OF THE ILLUSTRATED EMBODIMENTS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. Throughout the drawings, like-referenced characters are generally used to designate like elements.

Note: Throughout this disclosure, subscripted reference numbers (e.g., 10₁ or 10_{A}) may be used to designate multiple separate appearances of elements of a single species, whether in a drawing or not; for example: 10₁ is a single appearance (out of a plurality of appearances) of element 10. The same elements can alternatively be referred to without subscript (e.g., 10 and not 10₁) when not referring to a specific one of the multiple separate appearances, i.e., to the species in general. In some cases, subscripted reference numbers are used to designate an element of the same species having a different design but the same functionality as other elements of the same species.

For convenience, in the context of the description herein, various terms are presented here. To the extent that definitions are provided, explicitly or implicitly, here or elsewhere in this application, such definitions are understood to be consistent with the usage of the defined terms by those of skill in the pertinent art(s). Furthermore, such definitions are to be construed in the broadest possible sense consistent with such usage.

A bottle system according to embodiments comprises a bottle and a cap. The cap contains arrangements for use in a mist-delivery device, such as a piezo assembly comprising an ultrasonic mesh membrane, an electrical connection for powering the piezo assembly, and a one-way valve for introducing a liquid such as water or other aqueous liquid into the bottle system. The nebulizer arrangements of the cap are preferably configured to use vibrating mesh technology, as is known in the field of nebulizers, to expel, from the bottle assembly, an aerosol comprising fine droplets of whatever liquid is introduced into the bottle assembly. Fine droplets can be less than 50 microns in diameter, or less than 30 microns in diameter, or less than 20 microns in diameter or less than 10 microns in diameter or even finer. A mesh can be formed, for example, by using a laser to make uniform holes in a metal disk, or by any other known method.

A mist-delivery system according to embodiments comprises a bottle system as described herein, a housing such as a case or sleeve or housing, a fan, a power supply including (for example) a battery, and control circuitry for controlling the activation and operation of the device. The device is preferably configured to deliver, using the onboard piezo assembly, a mist of droplets comprising an aqueous liquid and, optionally, a biologically-active material in an admixture with the liquid. The mist departing the device can be entrained by an airflow generated by the fan so as to direct the mist in a desired direction and constrain its lateral dispersion.

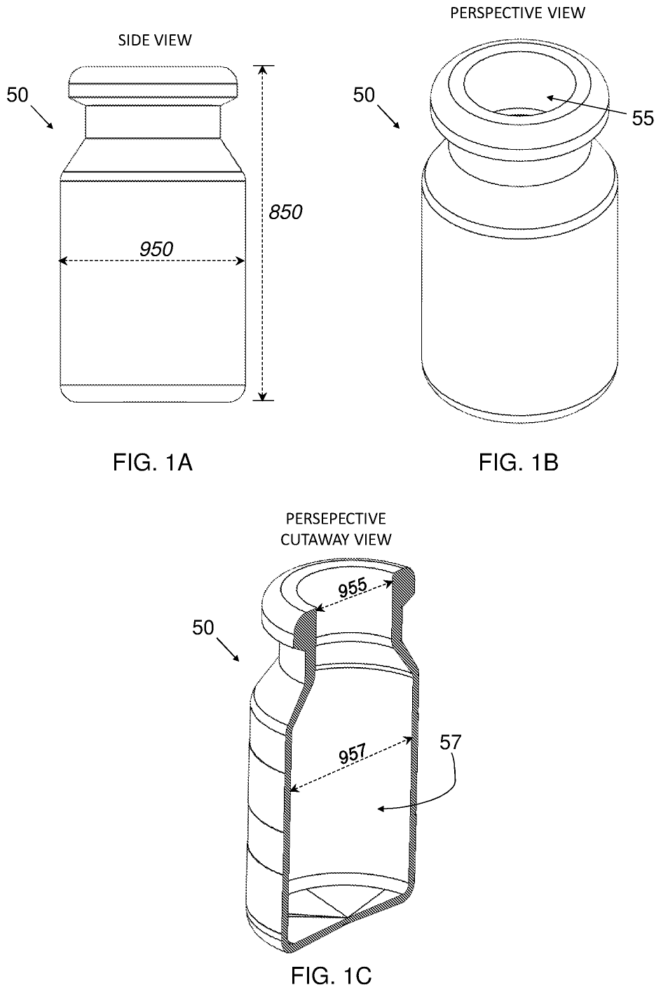

Referring now to the figures and in particular to FIGS. 1A, 1B and 1C, a bottle 50 includes an interior liquid-storage volume 57 and a neck-aperture 55. It can be desirable for the diameter 955 of the neck-aperture 55 to be smaller than a maximum diameter 957 of the liquid-storage volume 57. The bottle 50 can be made of any suitable material such as a metal or metal alloy, glass, or a plastic. In some embodiments, an outside diameter 950 of the bottle 50 can be as little as 1.5 cm and as much as 7.5 cm, and preferably between 2 cm and 5 cm, inclusive. A total external height 850 of the bottle 50 can be as little as 3 cm and as much as 12 cm, and preferably between 4 cm and 10 cm, inclusive.

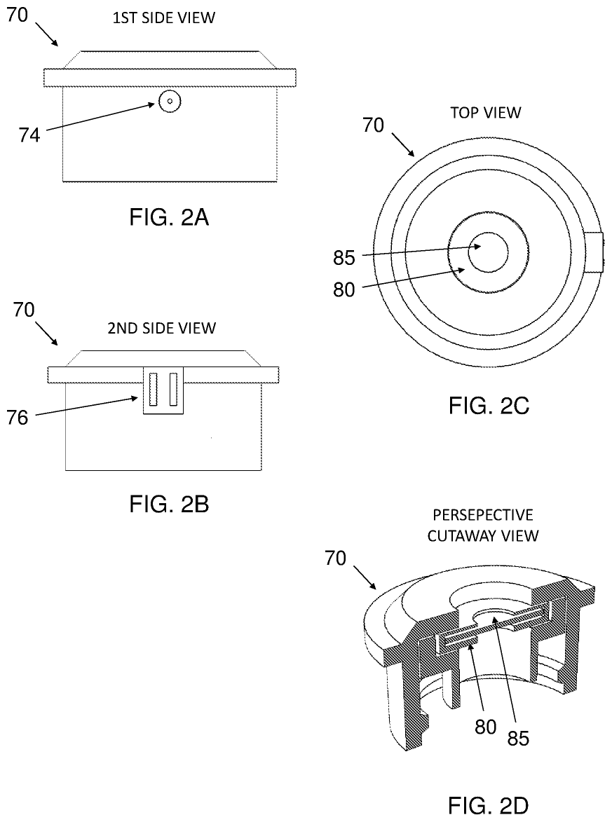

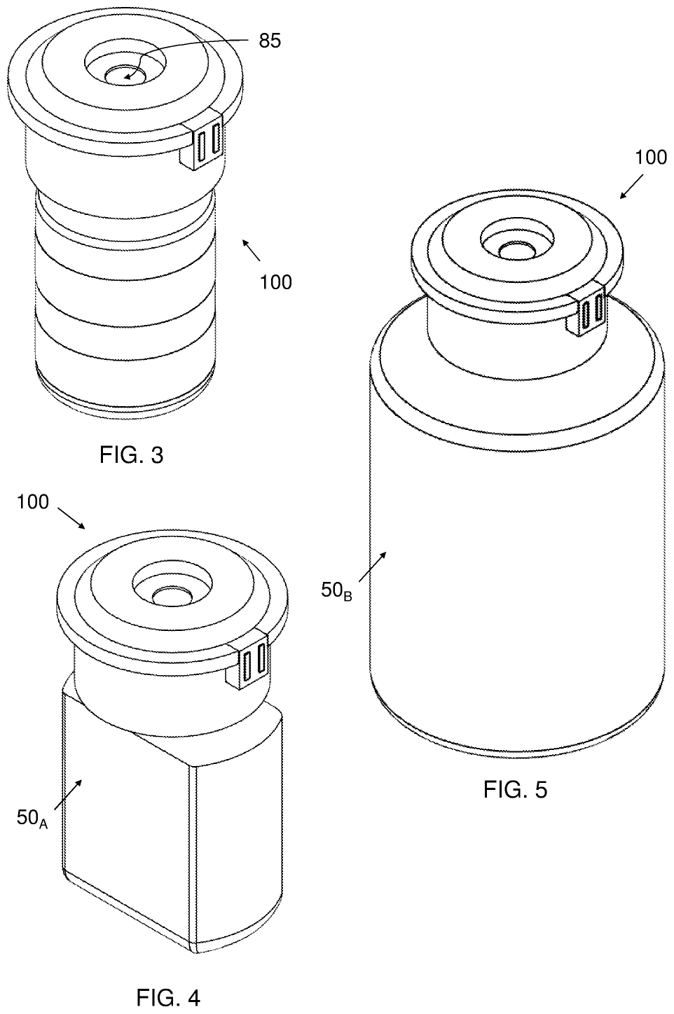

A cap 70 for sealing the bottle 50 is illustrated in FIGS. 2A, 2B, 2C and 2D. The term ‘sealing’ should be taken to understood that securing the cap 70 to the bottle 50 creates a liquid-tight (including watertight) seal between the cap 70 and the neck-aperture 55 of the bottle 50 so as to form a bottle system 100. It is noted that in preferred embodiments of the present invention, a bottle system 100 additionally comprises a capillary pathway as described hereinbelow including, inter alia, with respect to FIGS. 7-9. When a cap 70 is thus secured to the bottle 50, the resulting bottle system 100 may not allow a liquid stored in the bottle to 50 leak regardless of the orientation in which the bottle system 100 is held, as long as the bottle is held stationary. As shown in FIG. 2C, the cap 70 includes a piezo assembly 80 comprising an ultrasonic mesh membrane 85. Thus, if the bottle system 100 is shaken, some droplets of liquid may be forced by the shaking to leak out through the mesh membrane 85, rendering the bottle system 100 not watertight when not held stationary.

In embodiments, as illustrated in FIGS. 2A and 2B, a cap 70 can include a one-way conveyance 74 and an electrical contact 76. The one-way conveyance 74, or alternatively ‘one-way valve,’ is an arrangement that allows a user to introduce a liquid into the bottle system 100 after the cap 70 is secured to the bottle 50, but which does not allow the liquid to exit the bottle system 100 through the valve 74. An example of a suitable one-way valve is a duckbill valve available from Minivalve International of Cleveland, Ohio, USA. In an example, a bottle system 100 can be filled via the one-way valve 74 with a liquid that includes water and another substance such as a biologically active material such as, for example, an antioxidant material or composition. In another example, such a substance, in a solid phase, may be pre-disposed in a bottle system 100 when sold or distributed, or at anytime before securing a cap 70 to the bottle 50 of the bottle system 100, and a user can subsequently add an aqueous liquid, e.g., water or a dilute alcohol solution, to the bottle system 100 so as to create an admixture with the substance which is dissolved or suspended in the aqueous liquid. The position of the one-way conveyance 74 in FIG. 2A is shown schematically and can be anywhere on the cap 70 where the end of the one-way conveyance 74 that is inside the cap is in fluid communication with the interior liquid-storage volume 57 of the bottle 50. The position of the electrical contact 76 is also shown schematically. In some embodiments, the electrical contact can be on an outer surface of the bottle 50 rather than on the cap 70. The electrical contact 76 is connected, e.g., wired, to the piezo assembly 80 such that electric power delivered from outside the bottle system to the electrical contact will reach the piezo assembly 80 and activate the ultrasonic mesh membrane 85.

An assembled bottle system 100 using the bottle 50 of FIGS. 1A-C and cap 70 of FIGS. 2A-D is shown in FIG. 3. As illustrated in FIG. 3, the mesh membrane 85 is exposed to the atmosphere when the bottle system 100 is in this assembled state. Examples of alternatively-shaped bottles 50A, 50B, i.e., shaped differently than the bottle 50 of FIGS. 1A-C, are shown in FIGS. 4 and 5. Thus, the present invention is not tied to a specific size, shape or design of bottle other than the features described hereinabove.

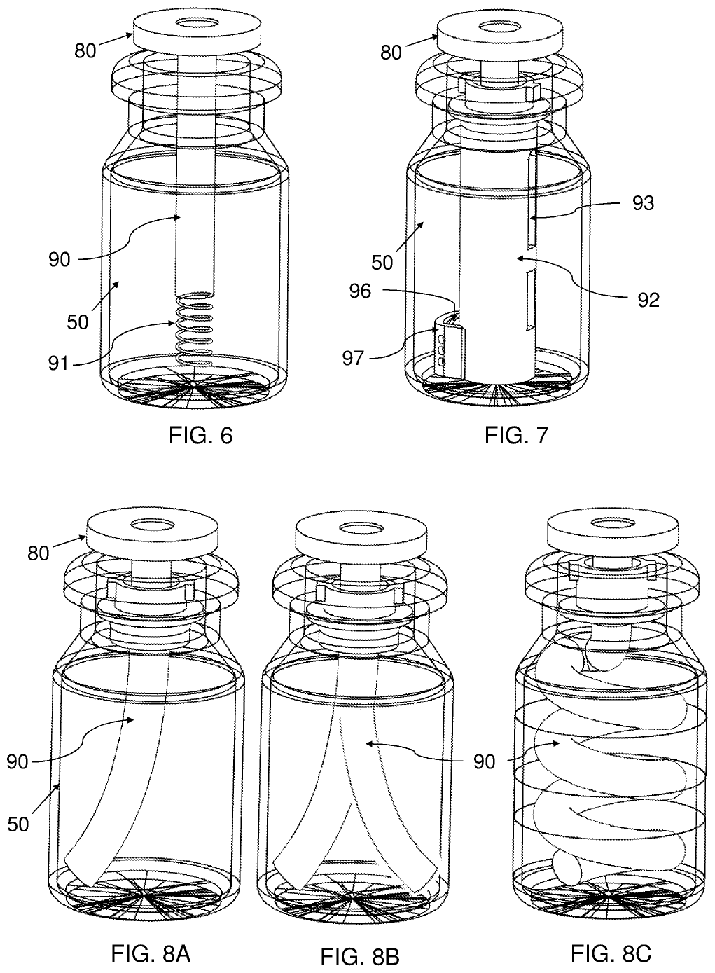

FIGS. 6, 7, 8A, 8B and 8C schematically illustrate examples of capillary pathways 90 suitable for use in bottle systems 100, each figure showing a largely ‘transparent’ 50 and the piezo assembly 80 onboard the cap 70 (which, besides the piezo assembly 80, is not shown in these figures). The capillary pathway 90 is typically disposed, and optionally held, so that a first portion thereof is in contact with the mesh membrane 85 of the piezo assembly 80, or displaced no more than 2 mm or no more than 1 mm from the mesh membrane 85. A second portion of the capillary pathway 90 is generally disposed within the liquid-storage volume 57 of a bottle 50 so as to establish a pathway for water transport from the liquid-storage volume 57 to the mesh membrane 85 installed in the cap 70. The first portion of the capillary pathway 90 can also be regarded as a ‘proximal portion’ and the second portion as a ‘distal portion’.

In preferred embodiments of the present invention the capillary pathway 90 is installed in the bottle system by the securing of a cap 70 to the aperture 55 of a bottle 50. A ‘capillary pathway’ 90 as the term is used herein is a material suitable for transport of water (or other aqueous liquid) along a pathway by capillary action. Such a material often includes fibers, such as plant-based fibers e.g., cellulose, polymer-based fibers e.g., polyester, glass fibers e.g., in a woven fabric or bundled or unbundled glass fibers, or carbon fibers. In some non-limiting examples, the fibers can be very small, i.e., having diameters in the range of several or tens of microns. In other examples, the fibers can be larger. While the term “pathway” may appear to imply that a pathway for water transport to a leak-alarm target may be a direct path, that is not necessarily the case. The transport of water through the capillary pathway may include progression in random directions or omnidirectional progression. In some embodiments, the capillary pathway 90 can include fibers arranged so as to form direct pathways from various parts of the liquid-storage volume 57 but this is not necessary for the capillary transport to be effective. The key in deploying the capillary pathway 90 is to ensure a substantially continuous pathway for the capillary transport regardless of either the direct nature of the transport or the fact that the water may be ‘spread’ in all directions throughout the capillary pathway material before reaching the target of the transport, i.e., the mesh membrane 85. In some embodiments, the capillary pathway can comprise a hydrophilic material that is effective to facilitate transport of water.

FIG. 6 illustrates a non-limiting example of a capillary pathway 90 with a biasing element 91 (illustrated in FIG. 6 by a spring element) which ensures that the capillary pathway 90 is kept in contact with the mesh membrane 85 of the piezo assembly 80.

FIG. 7 illustrates another non-limiting example in which the capillary pathway 90 (not visible in FIG. 7) is held in a preferred position within a holder 92 which has openings 93 that allow a liquid in the liquid-storage volume 57 to contact the capillary pathway 90 installed within. FIG. 7 also shows a quantity of a solid-phase substance 96 in a storage compartment 97, the substance 96 being provided in the liquid-storage volume 57 of the bottle 50 for later mixing with an aqueous liquid introduced thereinto and for being misted in an admixture with the aqueous liquid. The substance 96 can have any suitable solid-phase form factor, such as, without limitation, a powder, a tablet, or a capsule. In an example, the substance includes a biologically active substance such as an antioxidant. An antioxidant-containing mist delivered from the bottle system 100 can be used for inhalation and/for external use, e.g., on a user's skin. Examples of suitable antioxidant substances include, and not exhaustively, vitamins C and E, selenium, and carotenoids such as beta-carotene, lycopene, lutein, and zeaxanthin. In other examples, the substance 96 can include any substance suitable for inhalation or skin treatment.

As stated hereinabove, a capillary pathway 90 is provided so as to create a transport path for a liquid from the liquid-storage volume 57 of the bottle system 100 to the mesh membrane 85 onboard the cap 70. FIGS. 8A, 8B and 8C show a variety of non-limiting examples of capillary pathways 90 designed to be effective in various use cases of the bottle system 100. FIG. 8A shows an example in which the capillary pathway 90 is effective to transport liquid to the mesh membrane 85 when the bottle system is tipped, e.g., pivoted, to one side. In an example, a bottle system can be installed in a mist-delivery device configured to pivot the bottle system 100 in a specific direction such as the direction best served by the disposition of the capillary pathway 90 illustrated schematically in FIG. 8A. In another example, a bottle system can be installed in a mist-delivery device configured to pivot the bottle system 100 in either one of two specific opposing directions such as the two directions best served by the disposition of the capillary pathway 90 illustrated schematically in FIG. 8B. It will be obvious to the skilled artisan that a capillary pathway can be designed to support any number of potential pivoting directions and no additional examples need be illustrated. Another example of a capillary pathway 90 is shown schematically in FIG. 8C. As shown in FIG. 8C, the capillary pathway 90 can be designed so as to transport a liquid (e.g., to the mesh membrane 85) from practically any point within the water-storage volume 57 of a bottle system 100.

In embodiments, a device for delivering a mist includes a housing. A housing is preferably configured to have a bottle system 100 disposed therewithin, along with a fan for generating an airflow and a power source for activating the piezo assembly 85. The housing also preferably houses control circuitry for controlling the operation of the piezo assembly and the fan.

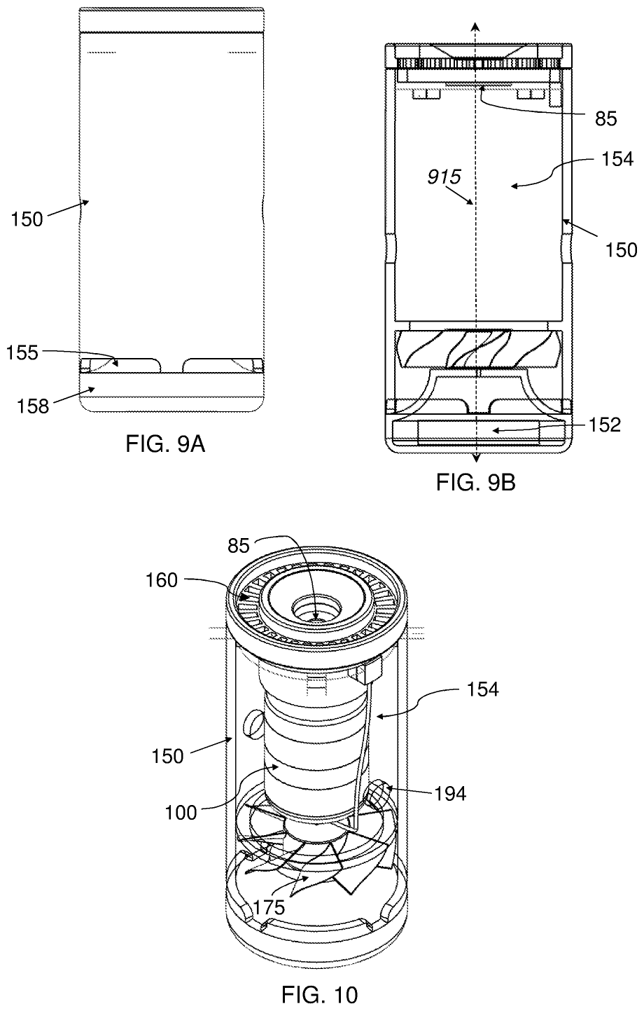

Reference is now made to FIGS. 9-12.

FIGS. 9A and 9B show a housing 150 adapted for use in a mist-delivery device. In this non-limiting example, a main portion of the housing 150 is formed as a cylindrical tube, although this is only for purposes of illustration and the housing can have any cross-section. The housing 150 can include a bottom section 158 which can be used to house a power supply 152 (e.g., a battery) and control circuitry (not visible). Openings 155 are placed to act as air inlets into the housing 150. FIG. 9B shows a central axis 915 which passes through both the fan 175 and the mesh membrane 85.

As can be seen in the schematically drawn perspective cutaway view of FIG. 10, a major portion of the housing 150 includes a plenum 154 in which a bottle system 100 is disposed. An annular air grille 160 at the top (i.e., the end opposite the bottom section 158) surrounds a central portion open to the mesh membrane 85 so as to enable delivery of a mist to the atmosphere outside the housing 150. A fan 175, which is preferably configured to be powered by the power source disposed within the bottom section, is disposed between the air-inlet openings 155 and the bottle system 100. The housing 150 also comprises an electrical contact corresponding to the electrical contact 76 of the cap 70, for delivering electricity from the power supply 152 to the piezo assembly 80 (via the electrical contact 76 of the cap 70) when the bottle system 100 is disposed within the plenum 154 of the housing 150 and fixedly (and, optionally, reversibly) held therein.

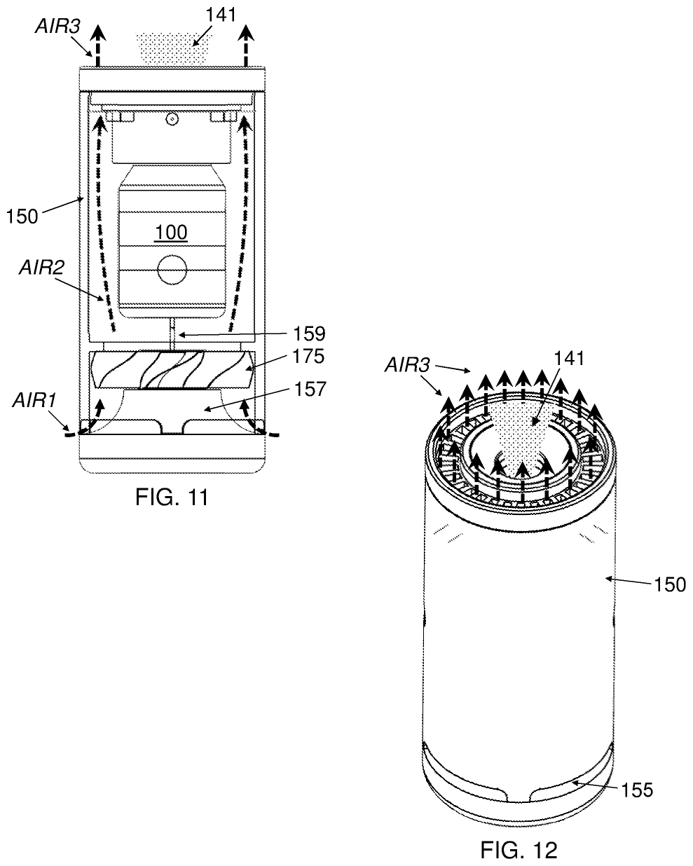

FIGS. 11 and 12 illustrate an airflow generated by the fan 175. In FIG. 11, the generated airflow is schematically divided into three segments: AIR1, AIR2, and AIR3. As shown in the cutaway drawing of FIG. 11, air (indicated by arrow AIR1) is drawn into the plenum 154 of the housing 150 through inlet openings 155. An air-directing element 157 can be provided to direct the incoming airflow segment AIR1 upwards. The fan 175 generates a positive pressure beyond it and a negative pressure behind it so as to draw in the AIR1 segment. As indicated by the arrow AIR2, the airflow segment AIR2 circumvents the bottle system 100 as it flows through the housing and toward the air grille 160 at the top of the housing 150. The fan-generated airflow exits the annular air grille 160 as indicated by the arrow AIR3. Mist 141, comprising droplets of a liquid stored in the liquid-storage volume 57 (optionally in an admixture with solid-phase substance 96, is delivered at the mesh membrane 85 into the atmosphere. As illustrated in FIG. 12, the cylindrical airflow segment AIR3 surrounds the mist 141. It is noted the airflow segment AIR3 is illustrated in FIG. 12 as cylindrical in accordance with the circular form factor of the annular air grille 160 of the exemplary housing 150 of FIG. 12. As stated hereinabove, the housing can have any shape, i.e., cross-section, which means that the air grille 160 can have different shapes as well (e.g., oval, elliptical, polygonal, etc.). In any case the air grille 160 will surround the mesh membrane 85 so that the generated airflow leaving the housing as airflow segment AIR3 surrounds the delivered mist 141 and the term ‘annular’ as used in this disclosure and in the claims appended thereto shall be understood to encompass such cases where the air grille 160 is not circular but nonetheless surrounds the mesh membrane 85. Moreover, such ‘surrounding’ for the purposes of this invention can encompass ‘surrounding with gaps’ and/or ‘partly surrounding’ as long as the mesh membrane 85 is at least more than 50% surrounded. Thus, an example of an annular surrounding air grille according to embodiments is an air grille 160 of any geographical shape, disposed around a majority of the periphery of the top of the housing 150.

According to embodiments, a user input device or element (not illustrated) such as, without limitation, a button, a slider, a switch or a touchscreen, can be used to activate both the fan 175 and the piezo assembly 80. The user input device or element can be disposed on an external surface of the housing 150, or elsewhere. Activation can be by completing an electrical circuit via electrical connection 159 which is provided for delivering electricity from the power supply 152 to the piezo assembly 80 and optionally to the fan 175. In some embodiments the fan 175 may be connected to the power supply 152 via a different connection (not shown). Upon activation, the fan 175 generates an airflow and the ultrasonic mesh membrane 85 delivers a mist 141 from a liquid stored in the liquid-storage volume 57 of the bottle system 100.

As the mist 141 begins to disperse upwards and outwards from the mesh membrane 85, the annular airflow entrains a portion of the mist 141. The entrainment has two effects: (i) since the airflow segment AIR3 is directable by directing, e.g., pivoting, the housing 150, the mist 141 is likewise directable in part or entirely together with the airflow segment AIR3, and (ii) lateral dispersion of the mist 141 is constrained by the airflow, meaning that less of the mist disperses laterally—outside of the surrounding airflow (e.g., the cylindrical airflow of FIG. 12)— than would be the case without the entrainment by airflow segment AIR3.

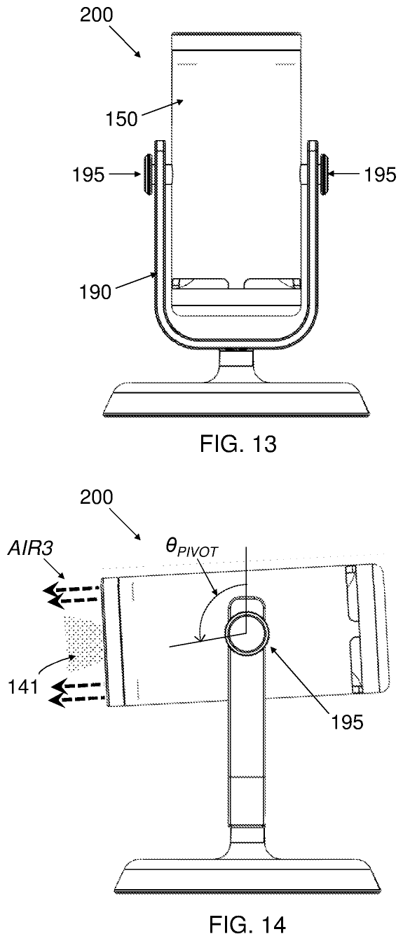

Reference is now made to FIGS. 13 and 14.

In embodiments, a mist-delivery device 200 includes a bottle system 100 and a housing 150 having a plenum 154 in which the bottle system 100 is disposed. In an assembled state, the bottle system 100 is securely, and optionally reversibly, held in a place designated for that purpose. The mist-delivery device 200 also includes control circuitry (not visible; as discussed hereinabove, said control circuitry can be disposed within a closed bottom section 158 of the housing 150 or anywhere else within the housing 150), and a base 190 for supporting the housing 150. The housing 150 preferably comprises a power supply 152, a powered fan 175, an air inlet 155 at a first end of the plenum 154, and an annular air grille 160 as an air outlet at a second end of the plenum 154; the inlet 155 and the outlet 160 defining an airflow path circumventing the replaceable bottle system 100. Note: as used in this disclosure and in the claims appended thereto, the terms ‘air inlet’ and ‘air outlet’ should be taken to mean any respective collection of one or more holes, slits, openings, grilles and the like; for example, an air inlet can include a first plurality of openings in a housing and an air outlet can include a second plurality of openings in the same housing, the two pluralities respectively displaced from each other as necessary to define an airflow path.

As shown in FIGS. 13 and 14, a base 190 can include one or more pivot elements 195 that enable pivoting thereabout of the housing 150. The housing can have corresponding pivot element receptors 194 (shown in FIG. 10) which physically connect with the pivot elements 195 of the base 190, so as to install the housing 150 in the base 190 and enable pivoting.

FIG. 14 illustrates an example of pivoting, in which the housing 150 is caused to pivot from an initial position at a vertical orientation (as shown in FIG. 13) to an angle θ_{PIVOT} from the vertical. As shown, the fan-generated airflow (shown as airflow segment AIR3) and the mist 141 entrained therewith are jointly directable by pivoting the housing 150. The pivot angle θ_{PIVOT} can be greater than 60°, or greater than 70°. In the non-limiting example of FIG. 14, θ_{PIVOT} has been drawn to be 100°. The capillary pathway 90 of the bottle system 100 of such mist-delivery devices 200 is configured to ensure water transport to the mesh membrane at these pivot angles down to a predetermined percentage of liquid remaining in the bottle system 100, e.g., down to 30% of capacity, down to 20% of capacity, or down to 10% of capacity. In some embodiments, the base 190 and the pivot element(s) are configured to allow pivoting away from the vertical orientation in a single direction, and in other embodiments, they are configured to enable pivoting in either of two opposing directions.

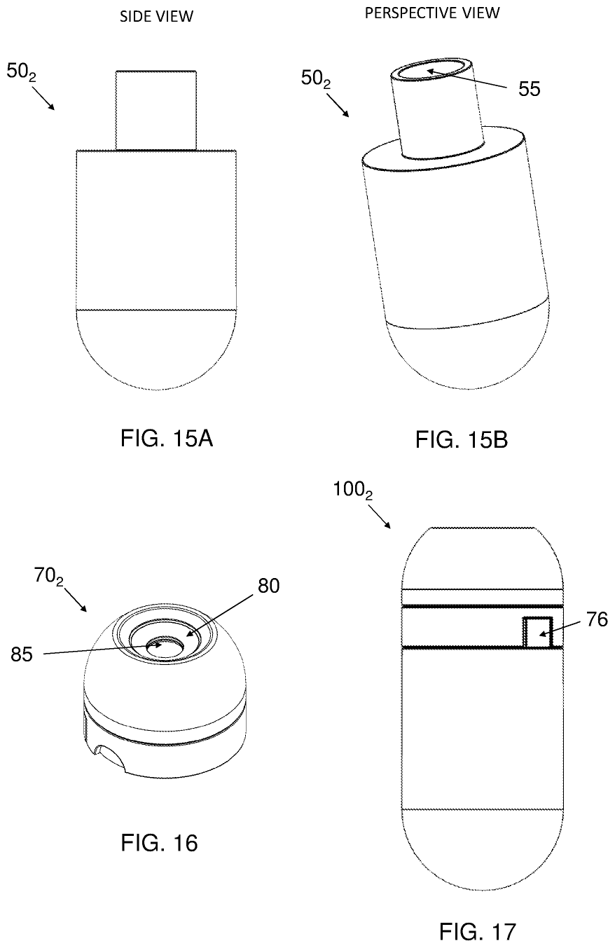

We now refer to FIGS. 15A-21B which show a bottle system and mist-delivery device similar in function to those of FIGS. 1A-14, with the bottle system, housing and base of the mist-delivery device all having a different aesthetic design.

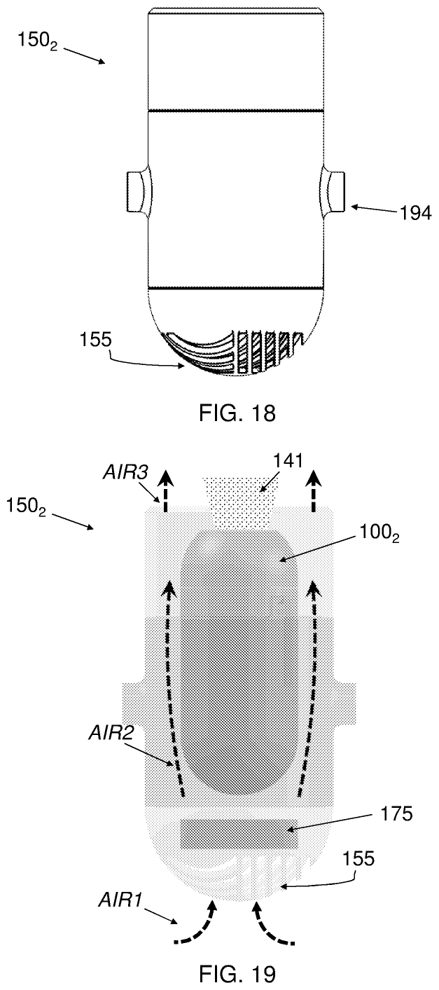

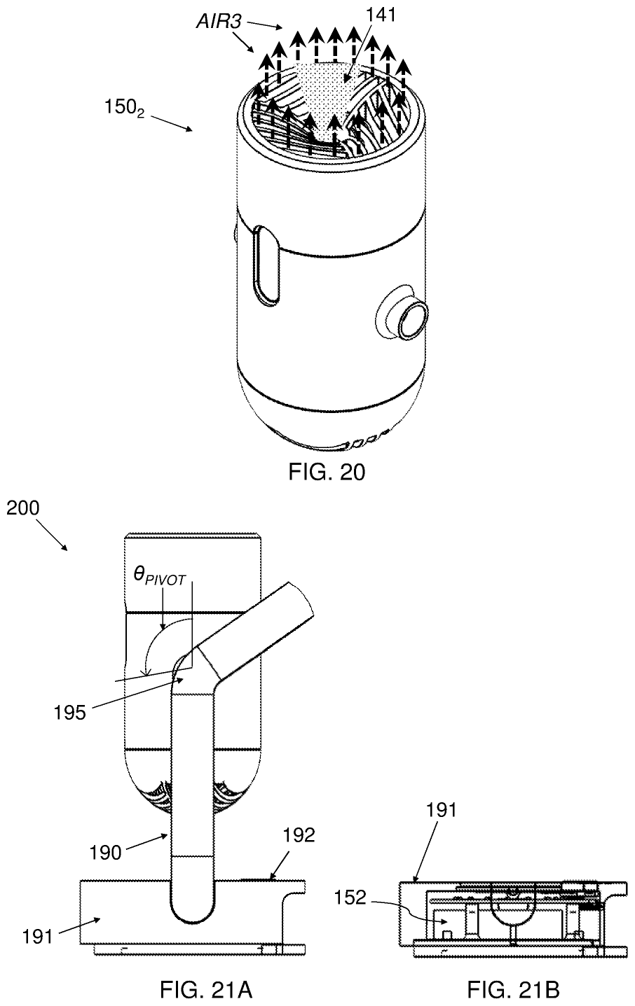

FIGS. 15A and 15B include views of a bottle 502 having a neck-aperture 55. FIG. 16 shows a cap 702 designed to complement the bottle 502 of FIGS. 15A and 15B. Similar to the cap 70 discussed with reference to 2A-C, cap 702 includes a piezo assembly 80 comprising an ultrasonic mesh membrane 85. As shown in FIG. 17, bottle 502 and cap 702 can be reversibly assembled to form a bottle system 1002. An electrical contact 76 is shown on an external surface of the cap 702—as was discussed hereinabove with respect to FIG. 2A. When assembled in a housing, the bottle system 1002 receives power through the electrical contact 76 from a matching contact (not shown) on the interior of housing 1502, which illustrated in FIG. 18. In housing 1502, the air inlet 155 in located on the bottom, as opposed to the design approach illustrated, for example, in FIG. 9A, where the air inlet 155 is located on the sides near the bottom. The cutaway drawing of housing 1502 is analogous to that of FIG. 19, in which the airflow generated by fan 175 is schematically divided into the same three segments of airflow: AIR1, AIR2, and AIR3. The functionality of the housing 1502 of FIG. 19 is the same as for housing 150 of FIG. 11, although in some embodiments, an air-directing element 157 may be rendered unnecessary with a bottom-inlet design such as in FIGS. 18-19. Similarly, FIG. 20 is analogous to FIG. 12, in which airflow segment AIR3 exiting the housing 1502 surrounds the mist 141 generated by the mesh membrane 85 (which is shown in FIG. 16). The mist delivery device 2002 of FIG. 21A, like mist-delivery device 200 of FIGS. 13-14, includes a housing 1502 installed on a base 190. The housing 1502 includes a bottle system 1002 and can be pivoted about pivot elements 195. As illustrated in FIG. 21A, the shape of the base 190 can be used to limit pivoting of the housing 1502 to one direction. In this design, as illustrated in FIG. 21B, the base 190 can include a bottom compartment 191 for a power supply 152, e.g., a battery (i.e., the power supply is not necessarily located within the housing 1502). The base 190 can also include user control(s) 192 for controlling the operation of the device 2002.

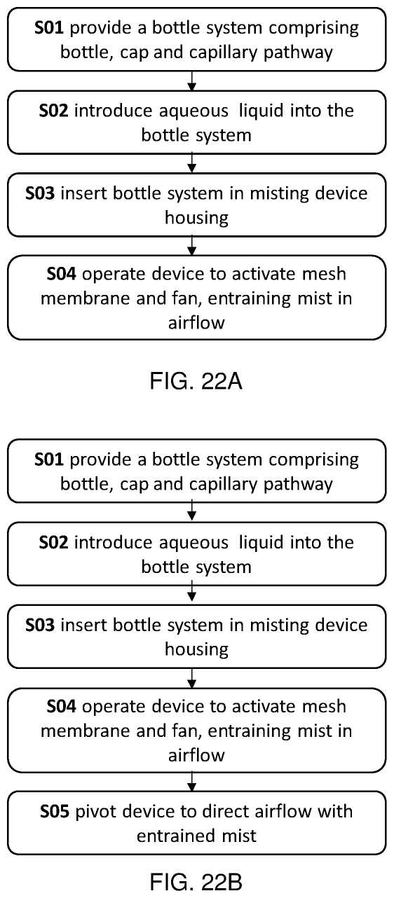

A method for non-thermal delivery of a mist 141 is disclosed. The method, as illustrated in the flowchart of FIG. 22A, can include the following steps:

Step S01, providing a bottle system 100 comprising bottle 50 having a liquid-storage volume 57, a cap 70 comprising a piezo assembly 80 including an ultrasonic mesh membrane 85, and a capillary pathway 90 for conveying a liquid by capillary action from the liquid-storage volume 57 to the mesh membrane 85 in accordance with any of the embodiments disclosed herein.

Step S02, introducing an aqueous liquid into the bottle 50 of the bottle system 100 through the unidirectional fluid conveyance (one-way valve) 74. In some embodiments, a quantity of a biologically active substance 96 in a solid phase, is disposed—prior to the introduction of the aqueous liquid—within the internal liquid-storage volume 57 of the provided bottle system 100. In such embodiments, the delivered mist 141 comprises droplets of an admixture of the biologically active substance 96 and the aqueous liquid.

Step S03, inserting the bottle system 100 into the plenum 154 of a housing 150 of a mist-delivery device 200. The housing 150 comprises a power supply 152, a powered fan 175, an air inlet 155 at a first end of the plenum 154, and an annular air outlet 160 at a second end of the plenum 154, the inlet 155 and outlet 160 defining an airflow path circumventing the inserted bottle system 100.

Step S04, operating the mist-delivery device 200 to deliver electricity from the power supply 152 to the fan 175 and to the piezo assembly 80, thereby causing the mesh membrane 85 to non-thermally deliver a mist 141 and causing the fan 175 to generate an airflow, fan-generated airflow exiting the annular air outlet 160 surrounds the mist 141, and is effective to entrain a portion of the delivered mist 141 and thereby constrain lateral dispersion of the mist 141.

In some embodiments, the method can include a fifth step, as illustrated by the flowchart in FIG. 22B:

Step S05, pivoting the mist-delivery device 200 to direct the airflow together with the entrained mist 141, e.g., as illustrated schematically in FIG. 14.

In some embodiments, not all of the steps recited in any of the methods are performed.

### Additional Discussion

According to embodiments, a non-thermal mist-delivery device comprises (a) a replaceable bottle system comprising (i) a bottle comprising an internal liquid-storage volume for holding a liquid, (ii) a cap comprising a piezo assembly including an ultrasonically vibrable mesh membrane, and (iii) a capillary pathway for conveying a portion of the liquid by capillary action from the liquid-storage volume to the mesh membrane; (b) a housing shaped to hold the replaceable bottle system therewithin, the housing comprising a fan, an air inlet and an annular air outlet, the inlet and the outlet defining an airflow path circumventing the replaceable bottle; and (c) control circuitry operative to electrically activate the fan and the piezo assembly in response to a user input, respectively to generate an airflow and to non-thermally deliver, via the aerosol outlet, a mist comprising droplets of the liquid, wherein the fan-generated airflow exiting the annular air outlet is effective to entrain a portion of the mist and thereby constrain lateral dispersion of the mist. The bottle system can additionally comprise a bottle-system-electrical-contact connected to the piezo assembly and disposed on an exposed surface of the bottle system. The bottle system can additionally comprise a base for supporting the housing. In some such embodiments, the base can comprise a pivot about which the housing can be caused to pivot through a pivot-range of at least 60°. The housing can additionally comprise an aerosol outlet. In some such embodiments, when the bottle system is stably held within the housing, the mesh membrane can face the aerosol outlet. The housing can additionally comprise a housing-electrical-contact. In some such embodiments, when the bottle system is stably held within the housing, the bottle-electric-contact can be in contact with the housing-electric-contact. The defined airflow path can pass through the fan. The mist-delivery device can additionally comprise a power supply for powering the fan and the piezo assembly. The bottle can additionally comprise a neck aperture for introducing a liquid therethrough into the liquid-storage volume at ambient pressure. The cap can comprise a fluid conveyance for introducing a liquid into the liquid-storage volume, the conveyance being configured to preclude egress of the liquid from the bottle.

In some embodiments, when the cap is secured to the bottle, a central axis of the bottle can pass through the mesh membrane. The cap can be configured to be secured to the bottle to form a liquid tight seal such that liquid can only leave the bottle via the bottle neck through the mesh. The capillary pathway can be attached to the cap such that its assembly in and/or disassembly from the bottle system is together with the cap. The bottle-electrical-contact can be disposed on an exposed surface of the cap. The portion of the mist entrained by the generated airflow can be directable by pivoting the mist-delivery device. The cap can be configured to be reversibly secured to the bottle. The securing of the cap to the bottle can create a waterproof seal between the cap and the neck aperture of the bottle. The disposition of the capillary pathway within the liquid-storage volume can be such that the mist-delivery device is effective, when the piezo assembly is electrically activated and the liquid-storage volume is at least 30% full, to deliver the mist throughout a pivot-range of at least 60°, or at least 70°. In some embodiments, when the bottle system is in an assembled state and the cap is secured to the bottle, a central axis of the bottle can pass through the mesh membrane. The housing can be caused to pivot through a pivot-range of at least 70°. The bottle can have a solid-phase biologically-active material disposed therewithin, which when dissolved or suspended in an aqueous liquid introduced into the liquid-storage volume through the fluid conveyance, is included in droplets of the delivered mist. The bottle can include a compartment for storing the biologically-active material, the compartment in fluid communication with the liquid-storage volume. The fan-generated airflow exiting the annular air outlet can surround the mist.

According to embodiments, a non-thermal mist-delivery device comprises: (a) a replaceable bottle system comprising (i) a bottle comprising an internal liquid-storage volume for holding a liquid, (ii) a cap comprising a piezo assembly including an ultrasonically vibrable mesh membrane, the cap configured to be secured to the bottle, (iii) a capillary pathway for conveying a portion of the liquid by capillary action from the liquid-storage volume to the mesh membrane; and (iv) a bottle-system-electrical-contact connected to the piezo assembly and disposed on an exposed surface of the bottle system; (b) a housing comprising a powered fan, an air inlet and an annular air outlet, an aerosol outlet, and a housing-electrical-contact, the housing shaped to stably hold the replaceable bottle system oriented therewithin such that the bottle-electric-contact is in contact with the housing-electric-contact, the mesh membrane faces the aerosol outlet, and the air inlet and annular air outlet collectively define an airflow path passing through the fan and circumventing the replaceable bottle; (c) a base for supporting the housing, the base comprising a pivot about which the housing can be caused to pivot through a pivot-range of at least 60′; and (d) control circuitry operative to electrically activate the fan and the piezo assembly in response to a user input, respectively to generate an airflow and to non-thermally deliver, via the aerosol outlet, a mist comprising droplets of the liquid held in the liquid-storage volume, wherein the fan-generated airflow exiting the annular air outlet is effective to entrain a portion of the mist and thereby constrain lateral dispersion of the mist. The mist-delivery device can additionally comprise a power supply for powering the fan and the piezo assembly. The bottle can additionally comprise a neck aperture for introducing a liquid therethrough into the liquid-storage volume at ambient pressure. The cap can comprise a fluid conveyance for introducing a liquid into the liquid-storage volume, the conveyance being configured to preclude egress of the liquid from the bottle. When the cap is secured to the bottle, a central axis of the bottle can pass through the mesh membrane. The cap can be configured to be secured to the bottle to form a liquid tight seal such that liquid can only leave the bottle via the bottle neck through the mesh. The capillary pathway can be attached to the cap such that its assembly in and/or disassembly from the bottle system is together with the cap. The bottle-electrical-contact can be disposed on an exposed surface of the cap. The portion of the mist entrained by the generated airflow can be directable by pivoting the mist-delivery device. The cap can be configured to be reversibly secured to the bottle. The securing of the cap to the bottle can create a waterproof seal between the cap and the neck aperture of the bottle. The disposition of the capillary pathway within the liquid-storage volume can be such that the mist-delivery device is effective, when the piezo assembly is electrically activated and the liquid-storage volume is at least 30% full, to deliver the mist throughout a pivot-range of at least 60°, or at least 70°. When the bottle system is in an assembled state and the cap is secured to the bottle, a central axis of the bottle can pass through the mesh membrane. The housing can be caused to pivot through a pivot-range of at least 70°. The bottle can have a solid-phase biologically-active material disposed therewithin, which when dissolved or suspended in an aqueous liquid introduced into the liquid-storage volume through the fluid conveyance, is included in droplets of the delivered mist. The bottle can include a compartment for storing the biologically-active material, the compartment in fluid communication with the liquid-storage volume. The fan-generated airflow exiting the annular air outlet can surround the mist. A bottle system can have any of the features disclosed hereinabove, in any combination.

A method of for non-thermal delivery of a mist is disclosed according to embodiments. The method comprises: (a) providing a bottle system comprising (i) a bottle having an internal liquid-storage volume, (ii) a cap secured to the bottle and comprising a piezo assembly including an ultrasonically vibrable mesh membrane, and (iii) a capillary pathway for conveying a liquid by capillary action from the liquid-storage volume to the mesh membrane; (b) introducing an aqueous liquid to the liquid-storage volume through a fluid conveyance configured to preclude egress of the liquid from the bottle; (c) inserting the bottle system into a plenum of a housing of a mist-delivery device, the device comprising a powered fan, an air inlet at a first end of the plenum, and an annular air outlet at a second end of the plenum, the inlet and outlet defining an airflow path circumventing the inserted bottle system; and (d) activating the device to deliver electricity from a power supply to the fan and to the piezo assembly, thereby causing the mesh membrane to non-thermally deliver a mist and causing the fan to generate an airflow, wherein the fan-generated airflow exiting the annular air outlet surrounds the mist, and is effective to entrain a portion of the delivered mist and thereby constrain lateral dispersion of the mist. It can be that (i) a quantity of a biologically active substance is disposed, in a solid phase, within the internal liquid-storage volume of the provided bottle system, and (ii) the delivered mist comprises droplets of an admixture of the biologically active substance and the aqueous liquid. The electricity delivered from the power supply to the piezo assembly can flow through an electrical contact disposed on an external surface of the cap of the bottle system. The method can additionally comprise directing the fan-generated airflow by pivoting the mist-delivery device on a support comprising a pivot. The disposition of the capillary pathway within the liquid-storage volume is such that the mist-delivery device can be effective, when the piezo assembly is electrically activated and the liquid-storage volume is at least 30% full, to deliver the mist throughout a pivot-range of at least 60°, or at least 70°.

According to embodiments, an externally-powered bottle system for use in a non-thermal mist delivery device comprises: (a) a bottle comprising an internal liquid-storage volume; (b) a cap configured for reversible engagement with the bottle, having a smaller volume than the bottle and comprising (i) a piezo assembly including an ultrasonically vibrable mesh membrane, and (ii) an exposed electrical contact connected to the piezo assembly for receiving electrical power from an external source to activate the piezo assembly, and (c) a capillary pathway for conveying a liquid by capillary action, wherein, when the bottle system is in an assembled state: (i) the cap is secured to the bottle so as to create a liquid-tight seal around the perimeter of the aperture and to position the electrical contact on an externally accessible surface of the assembled bottle system, and (ii) a proximal portion of the capillary pathway is restrained so as to be held in contact with an inwardly-facing surface of the mesh membrane or displaced therefrom by no more than 1 mm, and a distal portion of the capillary pathway is disposed within the liquid-storage volume, so that when a liquid is disposed in the liquid-storage volume, the capillary pathway is effective to convey a portion of the liquid to the mesh membrane for non-thermal production thereby of a mist comprising droplets of the liquid when the piezo assembly is electrically activated by delivery of electricity to the electrical contact. After the securing of the bottle system and in the absence of electricity delivery, it can be that the bottle system is water-tight when held in any orientation, and not water-tight when the bottle system is shaken. The mesh membrane can comprise a sub-50-micron mesh. In some embodiments, the mesh membrane can comprise a sub-30-micron mesh. In some embodiments, the mesh membrane can comprise a sub-10-micron mesh. The distal portion of the capillary pathway can be disposed so as to be in contact with a liquid disposed in the liquid-storage volume when the liquid-storage volume is at least 30% full and the bottle system is rotated from a vertical position by up to 60°, or up to 70°. The capillary pathway can be disposed within the bottle such that a center of a proximal-most 10% portion of the capillary pathway is closer to a central axis of the bottle than a center of a distal-most 10% portion of the capillary pathway. The cap can comprise a fluid conveyance having a one-way valve, provided such that when the cap is secured to the bottle, the conveyance can be effective to allow ingress of a liquid into the bottle and to preclude egress of the liquid from the bottle. The bottle can additionally comprise a neck aperture, and the securing of the cap to the bottle can create a water-tight seal between the cap and the neck-aperture. The cap can be reversibly secured to the bottle. When the bottle system is in an assembled state and the cap is secured to the bottle, a central axis of the bottle can pass through the mesh membrane. The bottle can include a solid-phase biologically-active material for being dissolved or suspended in droplets of an aqueous liquid misted by the piezo assembly. The can bottle can include a compartment for storing a solid-phase material, the compartment being in fluid communication with the liquid-storage volume.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons skilled in the art to which the invention pertains.

In the description and claims of the present disclosure, each of the verbs, “comprise”, “include” and “have”, and conjugates thereof, are used to indicate that the object or objects of the verb are not necessarily a complete listing of members, components, elements or parts of the subject or subjects of the verb. As used herein, the singular form “a”, “an” and “the” include plural references unless the context clearly dictates otherwise. For example, the term “a marking” or “at least one marking” may include a plurality of markings.

1. An externally-powered bottle system for use in a non-thermal mist delivery device, comprising:
a. a bottle comprising an internal liquid-storage volume and a neck-aperture;
b. a cap configured for reversible engagement with the bottle, the cap having a smaller volume than the bottle and comprising (i) a piezo assembly including a sub-50-micron-mesh ultrasonically-vibrable mesh membrane, and (ii) an exposed electrical contact connected to the piezo assembly for receiving electrical power from an external source to activate the piezo assembly; and
c. a capillary pathway for conveying a liquid by capillary action, wherein when the bottle system is in an assembled state:
i. the cap is secured to the bottle so as to position the electrical contact on an externally accessible surface of the assembled bottle system and to create a water-tight seal around the perimeter of the neck-aperture such that the bottle system is water-tight when held in any orientation, and not water-tight when the bottle system is shaken,
ii. a proximal portion of the capillary pathway is restrained so as to be held in contact with an inwardly-facing surface of the mesh membrane or displaced therefrom by no more than 1 mm, and
iii. a distal portion of the capillary pathway is disposed within the liquid-storage volume so as to be in contact with a liquid disposed in the liquid-storage volume, such that when the liquid-storage volume is at least 30% full and the bottle system is in a vertical position or rotated from a vertical position by up to 60°, the capillary pathway is effective to convey a portion of the liquid to the mesh membrane for non-thermal production thereby of a mist comprising droplets of the liquid when the piezo assembly is electrically activated by delivery of electricity to the exposed electrical contact.

2. The bottle system of claim 1, wherein the mesh membrane comprises a sub-30-micron mesh.

3. The bottle system of claim 1, wherein the mesh membrane comprises a sub-10-micron mesh.

4. The bottle system of any one of the preceding claims, wherein the capillary pathway is disposed within the bottle such that a center of a proximal-most 10% portion of the capillary pathway is closer to a central axis of the bottle than a center of a distal-most 10% portion of the capillary pathway.

5. The bottle system of any one of the preceding claims, wherein the cap comprises a fluid conveyance having a one-way valve, provided such that when the cap is secured to the bottle, the conveyance is effective to allow ingress of a liquid into the bottle and to preclude egress of the liquid from the bottle.

6. The bottle system of any one of the preceding claims, wherein when the bottle system is in an assembled state and the cap is secured to the bottle, a central axis of the bottle passes through the mesh membrane.

7. The bottle system of any one of the preceding claims, wherein the bottle includes a solid-phase biologically-active material for being dissolved or suspended in droplets of an aqueous liquid misted by the piezo assembly.

8. The bottle system of any one of the preceding claims, wherein the bottle includes a compartment for storing a solid-phase material, the compartment being in fluid communication with the liquid-storage volume.

9. The bottle system of one of the preceding claims, wherein the securing of the cap to the bottle is reversible.

10. The bottle system of claim 9, wherein the neck-aperture of the bottle and an inlet portion of the cap are correspondingly threaded such that securing the cap to the bottle can be accomplished by screwing one into the other.

11. The bottle system of claim 10, wherein removing the secured cap from the bottle can be accomplished without tools by applying a maximum torque of no more than 2.5 N·m.

12. The bottle system of claim 9, wherein the neck-aperture of the bottle and an inlet portion of the cap are configured to snap together so as to secure the cap to the bottle, at least one of the neck-aperture and the inlet portion including a snap-connector feature.

13. The bottle system of claim 9, wherein the neck-aperture of the bottle and an inlet portion of the cap, when in the assembled state, are reversibly held together by static friction.

14. The bottle system of either one of claim 12 or 13, wherein removing the secured cap from the bottle can be accomplished without tools by applying a maximum separating force of no more than 25 N.

15. A non-thermal mist-delivery device, comprising:
a. a replaceable bottle system comprising (i) a bottle comprising an internal liquid-storage volume for holding a liquid and a neck aperture for introducing a liquid therethrough into the liquid-storage volume at ambient pressure, (ii) a cap comprising a piezo assembly including an ultrasonically vibrable mesh membrane, the cap configured to be reversibly secured to the bottle to create a waterproof seal between the cap and the neck aperture of the bottle, (iii) a capillary pathway for conveying a portion of the liquid by capillary action from the liquid-storage volume to the mesh membrane, and (iv) a bottle-system-electrical-contact connected to the piezo assembly and disposed on an exposed surface of the bottle system;
b. a housing comprising a powered fan, an air inlet and an annular air outlet, an aerosol outlet, and a housing-electrical-contact, the housing shaped to stably hold the replaceable bottle system oriented therewithin such that the bottle-electric-contact is in contact with the housing-electric-contact and the mesh membrane faces the aerosol outlet;
c. a base for supporting the housing, the base comprising a pivot about which the housing can be caused to pivot through a pivot-range of at least 60′;
d. control circuitry operative to electrically activate the fan and the piezo assembly in response to a user input, respectively to generate an airflow and to non-thermally deliver, via the aerosol outlet, a mist comprising droplets of the liquid held in the liquid-storage volume; and
e. a power supply for powering the fan and the piezo assembly,
wherein when the bottle system is in an assembled state:
i. the cap is secured to the bottle so as to create a water-tight seal around the perimeter of the neck-aperture such that the bottle system is water-tight when held in any orientation, and not water-tight when the bottle system is shaken,
ii. a proximal portion of the capillary pathway is restrained so as to be held in contact with an inwardly-facing surface of the mesh membrane or displaced therefrom by no more than 1 mm, and
iii. a distal portion of the capillary pathway is disposed within the liquid-storage volume so as to be in contact with a liquid disposed in the liquid-storage volume, such that when the liquid-storage volume is at least 30% full and the bottle system is in a vertical position or rotated from a vertical position by up to 60°, the capillary pathway is effective to convey a portion of the liquid to the mesh membrane for non-thermal production thereby of a mist comprising droplets of the liquid when the piezo assembly is electrically activated by delivery of electricity to the exposed electrical contact.

16. The mist-delivery device of claim 15, wherein the neck-aperture of the bottle and an inlet portion of the cap are correspondingly threaded such that securing the cap to the bottle can be accomplished by screwing one into the other.

17. The mist-delivery device of claim 16, wherein removing the secured cap from the bottle can be accomplished without tools by applying a maximum torque of no more than 2.5 N·m.

18. The mist-delivery device of claim 15, wherein the neck-aperture of the bottle and an inlet portion of the cap are configured to snap together so as to secure the cap to the bottle, at least one of the neck-aperture and the inlet portion including a snap-connector feature.

19. The mist-delivery device of claim 18, wherein the neck-aperture of the bottle and an inlet portion of the cap, when in the assembled state, are reversibly held together by static friction.

20. The bottle system of either one of claim 18 or 19, wherein removing the secured cap from the bottle can be accomplished without tools by applying a maximum separating force of no more than 25 N.

21. The mist-delivery device of any one of claims 15 to 20, wherein the cap comprises a fluid conveyance for introducing a liquid into the liquid-storage volume, the conveyance being configured to preclude egress of the liquid from the bottle.

22. The mist-delivery device of any one of claims 15 to 21, wherein the capillary pathway is attached to the cap such that its assembly in and/or disassembly from the bottle system is together with the cap.

23. The mist-delivery device of any one of claims 15 to 22, wherein when the bottle system is in an assembled state and the cap is secured to the bottle, a central axis of the bottle passes through the mesh membrane.

24. The mist-delivery device of any one of claims 15 to 23, wherein the air inlet and annular air outlet collectively define an airflow path passing through the fan and circumventing the replaceable bottle.

25. The mist-delivery device of any one of claims 16 to 24, wherein the fan-generated airflow exiting the annular air outlet is effective to entrain a portion of the mist and thereby constrain lateral dispersion of the mist.

26. The mist-delivery device of any one of claims 16 to 25, wherein the portion of the mist entrained by the generated airflow is directable by pivoting the mist-delivery device.

27. The mist-delivery device of any one of claims 16 to 26, wherein the fan-generated airflow exiting the annular air outlet surrounds the mist.

28. The bottle system of any one of claims 15 to 27.

29. A method for non-thermal delivery of a mist, the method comprising:
a. providing a bottle system comprising (i) a bottle having an internal liquid-storage volume for holding a liquid, (ii) a cap comprising a piezo assembly including a sub-50-micron ultrasonically-vibrable mesh membrane, and (iii) a capillary pathway for conveying a liquid by capillary action from the liquid-storage volume to the mesh membrane;
b. introducing an aqueous liquid to the liquid-storage volume, at ambient pressure, through a neck-aperture of the bottle;
c. securing the cap to the bottle to create a water-tight seal between the cap and the neck aperture of the bottle such that the bottle system is water-tight when held in any orientation, and not water-tight when the bottle system is shaken;
d. inserting the bottle system into a plenum of a housing of a mist-delivery device, the mist-delivery device comprising (i) a powered fan, (ii) a power supply for powering the fan and the piezo assembly, (iii) a base for supporting the housing, the base comprising a pivot about which the housing can be caused to pivot through a pivot-range of at least 60°, (iv) control circuitry operative to electrically activate the fan and the piezo assembly in response to a user input;
e. activating the device to deliver electricity from a power supply to the fan and to the piezo assembly, thereby causing the mesh membrane to non-thermally deliver a mist and causing the fan to generate an airflow, and
f. directing the fan-generated airflow by pivoting the mist-delivery device on a support comprising a pivot,
wherein the bottle system is inserted in the housing in an assembled state such that:
i. a proximal portion of the capillary pathway is restrained so as to be held in contact with an inwardly-facing surface of the mesh membrane or displaced therefrom by no more than 1 mm, and
ii. a distal portion of the capillary pathway is disposed within the liquid-storage volume so as to be in contact with a liquid disposed in the liquid-storage volume, such that when the liquid-storage volume is at least 30% full and the bottle system is in a vertical position or rotated from a vertical position by up to 60°, the capillary pathway is effective to convey a portion of the liquid to the mesh membrane for non-thermal production thereby of a mist comprising droplets of the liquid when the piezo assembly is electrically activated.

30. The method of claim 29, wherein the neck-aperture of the bottle and an inlet portion of the cap are correspondingly threaded such that securing the cap to the bottle can be accomplished by screwing one into the other.

31. The method of claim 29, wherein the neck-aperture of the bottle and an inlet portion of the cap are configured to snap together so as to secure the cap to the bottle, at least one of the neck-aperture and the inlet portion including a snap-connector feature.

32. The method of claim 29, wherein the neck-aperture of the bottle and an inlet portion of the cap, when in the assembled state, are reversibly held together by static friction.

33. The method of any one of claims 29 to 32, wherein the housing comprises an air inlet at a first end of the plenum, and an annular air outlet at a second end of the plenum, the inlet and outlet defining an airflow path circumventing the inserted bottle system.

34. The method of either one of claim 32 or 34, wherein the electricity delivered from the power supply to the piezo assembly flows through an electrical contact disposed on an external surface of the cap of the bottle system.

35. The method of any one of claims 29 to 33, wherein the fan-generated airflow exiting the annular air outlet surrounds the mist, entrains a portion of the delivered mist and thereby constrains lateral dispersion of the mist.

36. The method of any one of claims 29 to 35, additionally comprising: pivoting the mist-delivery device to direct the portion of the mist entrained by the generated airflow.

37. A non-thermal mist-delivery device, comprising:
a. a replaceable bottle system comprising (i) a bottle comprising an internal liquid-storage volume for holding a liquid, and (ii) a cap comprising a piezo assembly including an ultrasonically vibrable mesh membrane,
b. a housing shaped to hold the replaceable bottle system therewithin, the housing comprising a fan, an air inlet and an annular air outlet, the inlet and the outlet defining an airflow path circumventing the replaceable bottle; and
c. control circuitry operative to electrically activate the fan and the piezo assembly in response to a user input, respectively to generate an airflow and to non-thermally deliver, via the aerosol outlet, a mist comprising droplets of the liquid.

38. The mist-delivery device of claim 37, wherein the cap is configured to be secured to the bottle.

39. The mist-delivery device of either one of claim 37 or 38, additionally comprising a power supply for powering the fan and the piezo assembly.

40. The mist-delivery device of any one of claims 37 to 39, wherein the replaceable bottle system additionally comprises a capillary pathway for conveying a portion of the liquid by capillary action from the liquid-storage volume to the mesh membrane.

41. The mist-delivery device of any one of claims 37 to 40, wherein the fan-generated airflow exiting the annular air outlet is effective to entrain a portion of the mist and thereby constrain lateral dispersion of the mist.

42. The mist-delivery device of any one of claims 37 to 41, wherein the replaceable bottle system additionally comprises a bottle-system-electrical-contact connected to the piezo assembly and disposed on an exposed surface of the bottle system.

43. The mist-delivery device of any one of claims 37 to 42, wherein the housing additionally comprises a housing-electrical-contact, and the housing is shaped to stably hold the replaceable bottle system oriented therewithin such that the bottle-electric-contact is in contact with the housing-electric-contact and the mesh membrane faces the aerosol outlet.

44. The mist-delivery device of any one of claims 37 to 43, additionally comprising a base for supporting the housing, the base comprising a pivot about which the housing can be caused to pivot through a pivot-range of at least 60°.

45. The mist-delivery device of any one of claims 37 to 44, additionally comprising control circuitry operative to electrically activate the fan and the piezo assembly in response to a user input, respectively to generate an airflow and to non-thermally deliver, via the aerosol outlet, a mist comprising droplets of the liquid held in the liquid-storage volume.

46. The mist-delivery device of any one of claims 37 to 45, wherein when the bottle system is in an assembled state and the cap is secured to the bottle, a central axis of the bottle passes through the mesh membrane.
